(19) United States
(12) Patent Application Publication
(10) Pub. No.: US 2013/0296902 A1
(43) Pub. Date: Nov. 7, 2013

(54) VACUUM ANCHORING CATHETER

(71) Applicants: Carlos Vonderwalde, Richmond (CA); Amir Miller, Richmond (CA)

(72) Inventors: Carlos Vonderwalde, Richmond (CA); Amir Miller, Richmond (CA)

(73) Assignee: Britamed Incorporated, Richmond (CA)

(21) Appl. No.: 13/825,291

(22) PCT Filed: Sep. 20, 2011
(86) PCT No.: PCT/CA11/01056

§ 371 Date: Jul. 23, 2013

Related U.S. Application Data
(60) Provisional application No. 61/384,692, filed on Sep. 20, 2010.

(51) Int. Cl. A61B 17/22; A61M 25/04

(52) U.S. Cl. CPC A61B 17/22; A61M 25/04
USPC 606/159

(57) ABSTRACT

Provided is a method for the treatment of blood vessel occlusions, comprising the localized anchoring of a catheter during the procedure by temporarily adhering its tip to the occlusion treatment site using a vacuum. Also provided is a catheter with a vacuum anchoring tip controlled by an externally generated vacuum, a catheter with a vacuum anchoring tip controlled by a self-generated vacuum, and a catheter with a vacuum anchoring tip in which the vacuum is controlled by an electronic signal. The localized anchoring method utilizes a vacuum to secure the tip of the catheter in place while allowing a free passage for the wire or dedicated occlusion penetrating device, and thereby frees the operator from constantly monitoring the tip position and pushing the catheter to support the advancement of the wire.

### TECHNICAL FIELD

The present disclosure relates in general to angioplasty, and in particular to methods and apparatus for use in the treatment of blood vessel occlusion, including chronic total occlusion.

### BACKGROUND

The treatment of blood vessel occlusions generally involves the use of percutaneous angioplastic techniques to advance a micro guiding catheter to the location of the occlusion, and to penetrate the occlusion with a wire or dedicated occlusion penetrating device in order to create a micro channel into which the operator can later introduce other percutaneous devices such as angioplasty balloons, and to fully restore blood flow. The mechanism behind occlusion crossing is based on a constant advancement of the wire or dedicated occlusion penetrating device, which allows it to be diverted into the natural micro-channels located within the occlusion until full crossing is achieved.

Blood vessel occlusions may be acute or chronic, and chronic occlusions, often referred to as Chronic Total Occlusion (“CTO”), are typically fibrotic and often also calcified. CTOs may also be longer than acute occlusions. Accordingly, relatively high axial forces may be required in order to penetrate and advance a wire or dedicated penetrating device through a CTO.

There is an obvious mechanical limitation to the amount of forward axial force that can be transmitted through a wire because a wire will easily buckle without radial support. Micro guiding catheters (which typically comprise a tight tube having an inner diameter that is only marginally greater than the diameter of the wire, and which are stiff but flexible enough to allow the operator to push them trough the vasculature of the patient to the CTO site) are accordingly commonly used in known CTO treatment techniques.

However, although the use of a micro guiding catheter improves the amount of available axial force, it does not provide the operator with the full potential of force delivery. This derives from the action-reaction physical law, as pushing a wire constrained within a tube against an obstacle will result in a force acting at the opposite direction from the obstacle back to the wire and to the constraining tube. If the constraining tube is dislodged from the treatment site, the wire in the vicinity of the dislodgement may be exposed, and thereby the wire may lose its ability to deliver axial force or buckle.

In order to keep the wire fully protected throughout the procedure, the operator must accordingly pay constant attention to the catheter's tip position, keeping it as close as possible to the occlusion. This is not, however, always feasible because the tortuous path the catheter may be required to follow to arrive at the treatment site can cause a loss of force and/or control at each of the bends the catheter makes. Additionally, in using a typical micro guiding catheter, the operator needs to be careful not to exceed the maximum allowed axial force that could result in buckling of the catheter itself.

Current state of the art micro guiding catheters thus provide a partial solution for wire buckling and thereby increase slightly the amount of force the operator can apply, but they do not contemplate catheter tip securement, and therefore do not provide the operator with the full potential of force transmutation through the wire. Other state of the art techniques have accordingly been developed to facilitate securement of the micro catheter at the occlusion treatment site.

These methods involve the use of an angioplasty balloon that, upon inflation, pushes the distal end of the micro guiding catheter shaft against the blood vessel wall. The shaft is therefore pressed between the inflated balloon and the vessel wall, and this keeps the distal end of the catheter relatively secured. However, the use of an angioplasty balloon to secure the distal end of a micro catheter has several disadvantages as well. Most important among these is the safety issue of pushing the shaft into a vessel wall, which could potentially cause serious injury. A further drawback is the resulting inability for the operator to reposition the catheter tip during the procedure since the catheter is virtually locked against the vessel wall. A variant of this method involves a coaxial set up that allows free movement of the wire; however, the risk of vessel injury due to balloon force applied is still present.

### SUMMARY

This summary is not an extensive overview intended to delineate the scope of the subject matter that is described and claimed herein. The summary presents aspects of the subject matter in a simplified form to provide a basic understanding thereof, as a prelude to the detailed description that is presented below.

Provided herein is a method for the treatment of blood vessel occlusions, comprising the localized anchoring of a catheter during the procedure by temporarily adhering its tip to the occlusion treatment site using a vacuum. Also provided is a catheter with a vacuum anchoring tip controlled by an externally generated vacuum, a catheter with a vacuum anchoring tip controlled by a self-generated vacuum, and a catheter with a vacuum anchoring tip in which the vacuum s controlled by an electronic signal. The localized anchoring method utilizes a vacuum to secure the tip of the catheter in place while allowing a free passage for the wire or dedicated occlusion penetrating device, and therby frees the operator from constantly monitoring the tip position and pushing the catheter to support the advancement of the wire.

### BRIEF DESCRIPTION OF THE DRAWINGS

For a fuller understanding of the nature and advantages of the disclosed subject matter, as well as the preferred mode of use thereof, reference should be made to the following detailed description, read in conjunction with the accompanying drawings. In the drawings, like reference numerals designate like or similar steps or components.

FIG. 1 is a schematic illustration of the prior art treatment of a blood vessel occlusion using a conventional micro guiding catheter, and showing the diversion of the wire or a dedicated occlusion penetrating device into the natural micro-channels located within the occlusion.

FIG. 2 is a schematic illustration of the prior art treatment of a blood vessel occlusion using a conventional micro guiding catheter, and showing the effects of the application of a forward axial force on an unsupported wire “without support”, and on a wire that is supported by a micro guiding catheter “with support”.

FIGS. 3 and 4 are schematic illustrations of the prior art treatment of a blood vessel occlusion using a conventional micro guiding catheter, and showing the dislodgement of the micro guiding catheter from the treatment site by virtue of the law of action-reaction.

FIG. 5 is a schematic illustration of the prior art treatment of a blood vessel occlusion using a conventional micro guiding catheter, and showing buckling of the wire in the vicinity of the dislodgement.

FIGS. 6 and 7 are schematic illustrations of a generalized embodiment of a vacuum anchoring tip for temporarily adhering the tip of a catheter to an occlusion site.

FIGS. 8 and 9 are cross-sectional views of a vacuum anchoring tip in accordance with embodiments of the present subject matter.

FIGS. 10 and 11 are perspective views of vacuum anchoring tips in accordance with embodiments of the present subject matter.

FIGS. 12-17 are cross-sectional views of a vacuum anchoring tip in accordance with embodiments of the present subject matter.

FIG. 18 is a schematic illustration a single chamber suction device.

FIG. 19 is a schematic illustration comparing a prior art single chamber suction device with a vacuum anchoring tips in accordance with embodiments of the present subject matter.

FIG. 20 is a cross-sectional view of a vacuum anchoring tip in accordance with embodiments of the present subject matter.

FIGS. 21-24 are partial perspective views of a catheter in accordance with embodiments of the present subject matter.

FIG. 25 is a cross-sectional view of a vacuum anchoring tip in accordance with an alternate embodiment of the present subject matter.

FIG. 26 is an enlarged perspective view of a spring frame of the vacuum anchoring tip of FIG. 25.

FIG. 27 is an exploded perspective view of 5 the vacuum anchoring tip of FIG. 25.

### DETAILED DESCRIPTION OF PREFERRED EMBODIMENTS

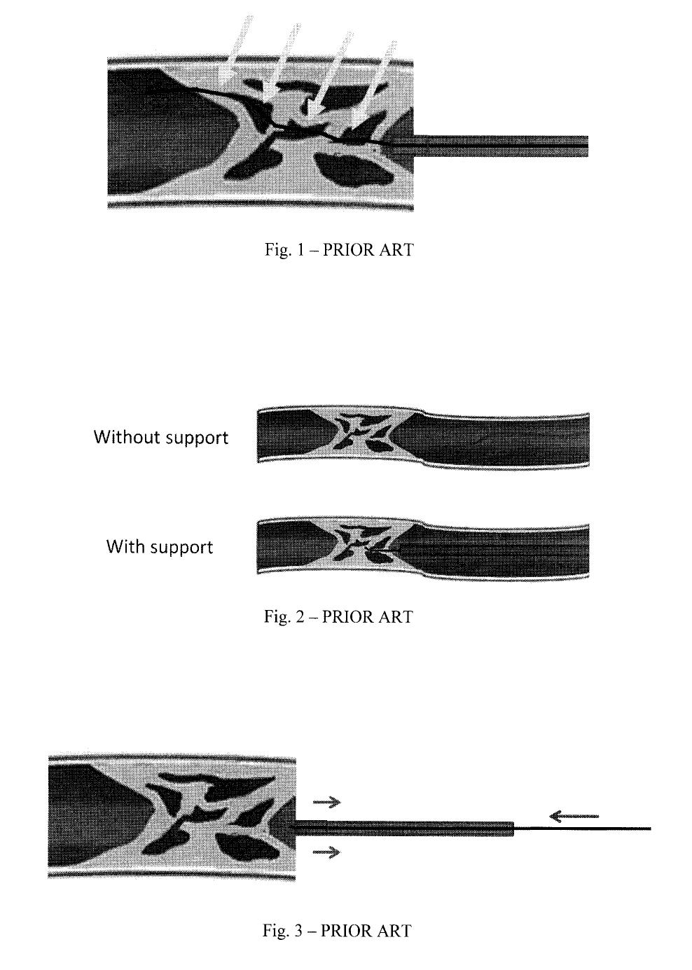

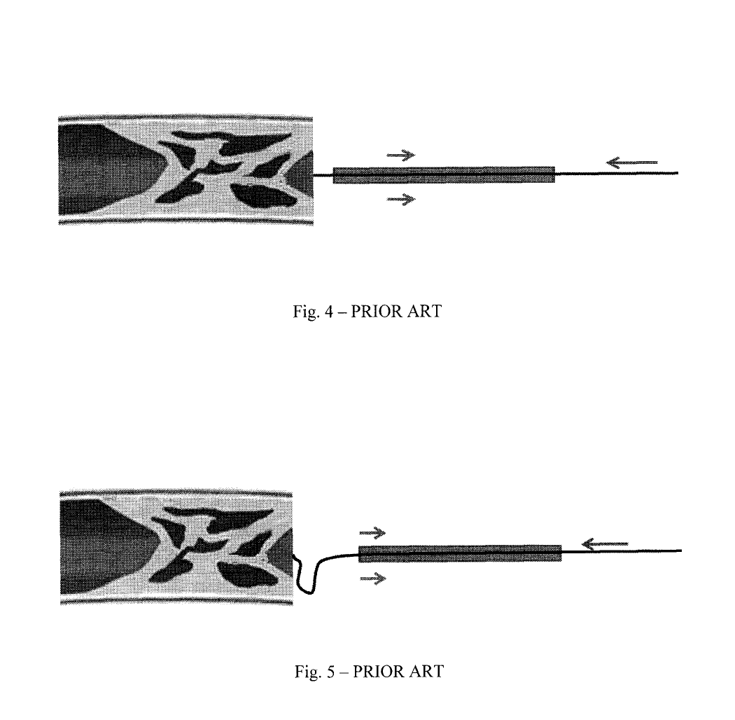

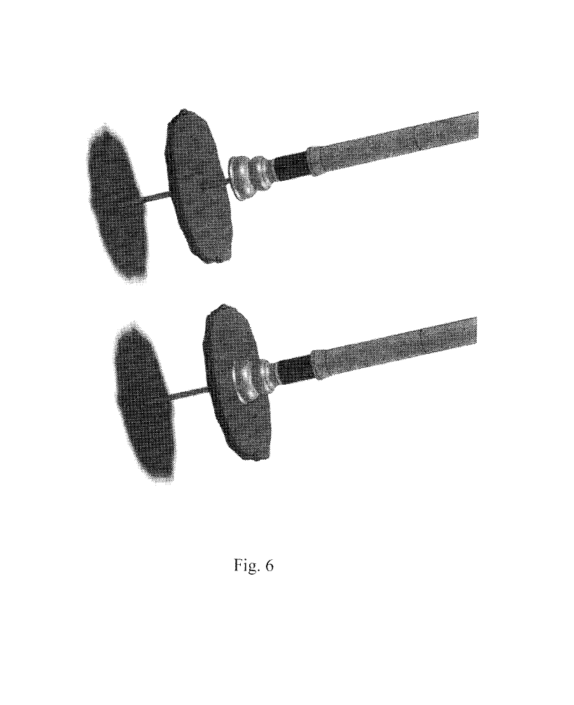

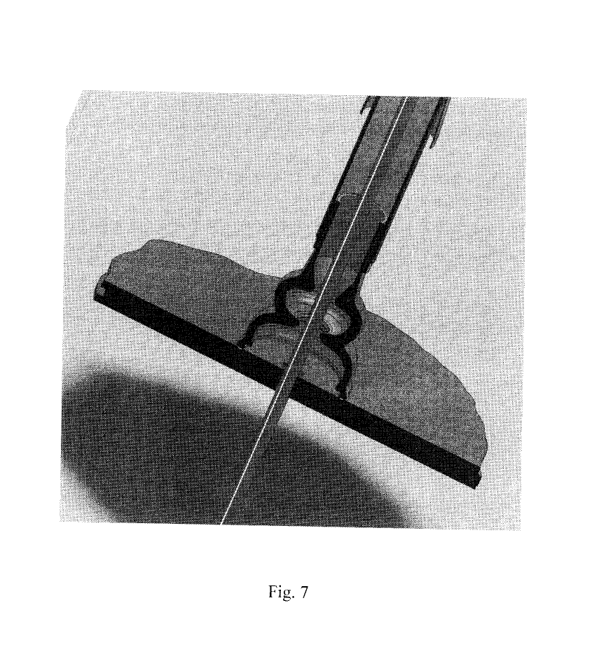

FIGS. 1 through 5 illustrate the prior art treatment of a blood vessel occlusion such as a CTO using a conventional micro guiding catheter, as discussed in the background section above. FIG. 1 illustrates the diversion of the wire or dedicated occlusion penetrating device into the natural micro-channels located within the occlusion. FIG. 2 shows the effects of the application of a forward axial force on an unsupported wire “without support”, and on a wire that is supported by a micro guiding catheter “with support”. FIGS. 3 and 4 show the dislodgement of the micro guiding catheter from the treatment site by virtue of the law of action-reaction, and FIG. 5 shows the resulting buckling of the wire in the vicinity of the dislodgement.

With reference to FIGS. 6 and 7, there is illustrated a generalized embodiment of a vacuum anchoring tip for temporarily adhering the tip of a catheter to an occlusion site. The vacuum may be externally generated or self generated, and may be controlled mechanically or by way of an electronic signal.

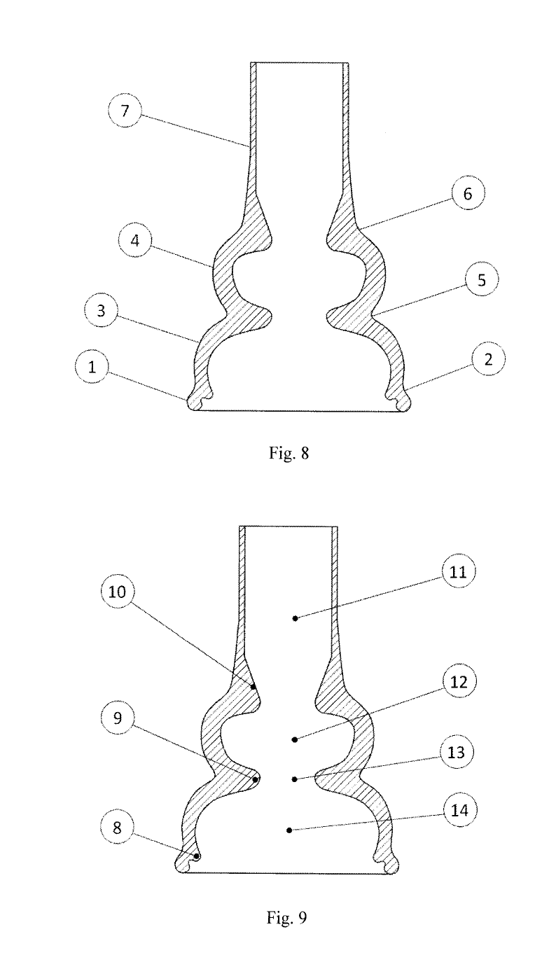

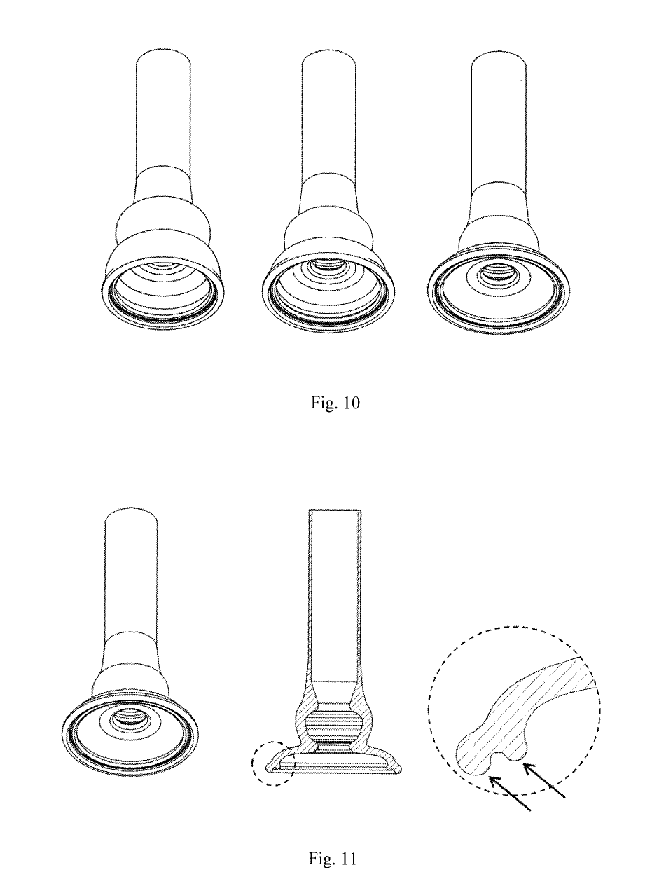

FIGS. 8 and 9 schematically illustrate in cross-section a vacuum anchoring catheter tip wherein the vacuum is created and controlled by an externally generated vacuum. The catheter is dimensioned to deliver a conventional guidewire, stiff wire or dedicated occlusion penetrating device through a firmly anchored tip to a blood vessel occlusion, and relies on a vacuum to secure the tip at the site of the vessel occlusion while allowing a free passage for the wire.

The tip 100 is preferably formed from a single piece of a flexible material that can be manufactured by injection molding, by two piece mold assembly methods, or by machining. In preferred embodiments, the outer surface geometry of tip 100 has seven distinct areas, as follows: sealing ring 1, sealing ring recess 2, contact chamber wall 3, vacuum chamber wall 4, chambers divider recess 5, vacuum chamber recess 6, and tail wall 7. The inner surface geometry of tip 100 also has, in preferred embodiments, seven distinct areas, as follows: secondary sealing ring 8, chambers divider septum 9, guiding cone 10, tail 11, vacuum chamber 12, chambers divider lumen 14, and contact chamber 14.

The sealing ring 1 serves as the primary contact zone for adhering the tip to the occlusion site to create an initial seal and thus to allow vacuum to be built up in the tip 100. Associated sealing ring recess 2 facilitates the sealing of the sealing ring 1 by enhancing the flexibility thereof vis-à-vis the occlusion site.

As vacuum is built up within tip 100, contact chamber 14 becomes the main interface between the tip 100 and the target surface of the occlusion site. Secondary sealing ring 8 is optional, and in embodiments that include it enhances further sealing ability of the contact chamber 14 by providing additional reinforcement.

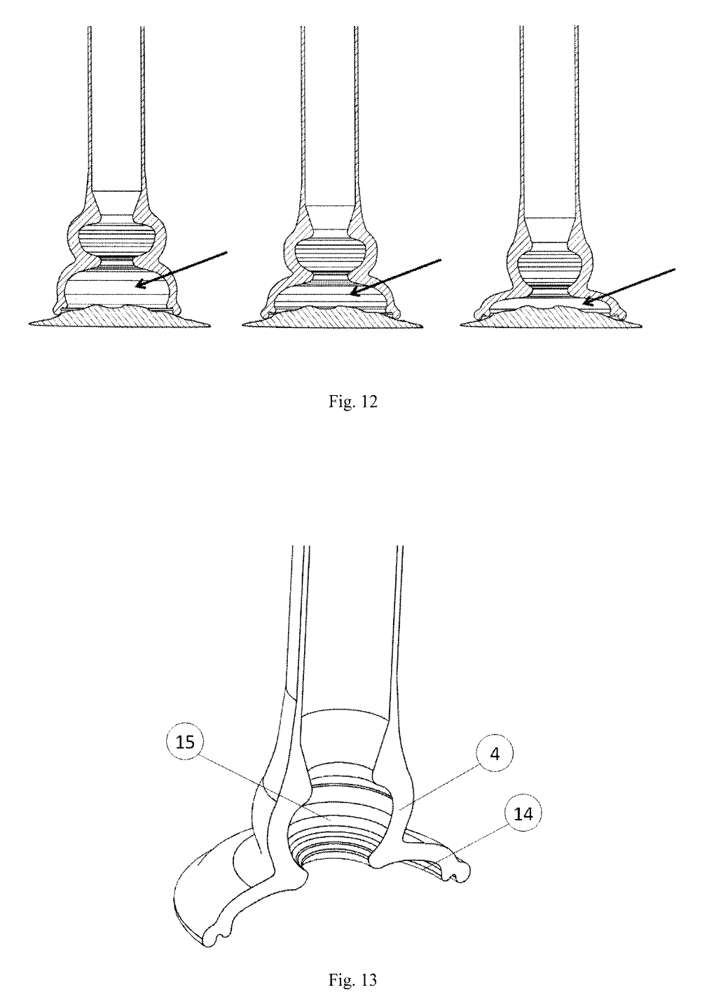

As is best seen in FIG. 12, the contact chamber 14 maintains a selected degree of vacuum during use, and is able to stretch to fit the topography of the target surface area whether it has rough, bumpy or smooth areas. To facilitate this, the wall 3 of contact chamber 14 may be thinner compared to other areas of the tip 100 to enhance the ability thereof to stretch, expand and generally accommodate for the target surface topography. The wall 3 of contact chamber 14 may also be manufactured from a lower durometer material to further assist in achieving these attributes.

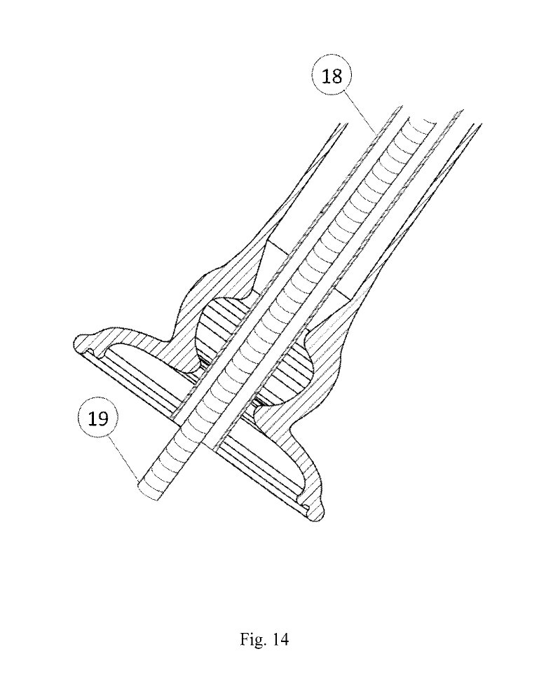

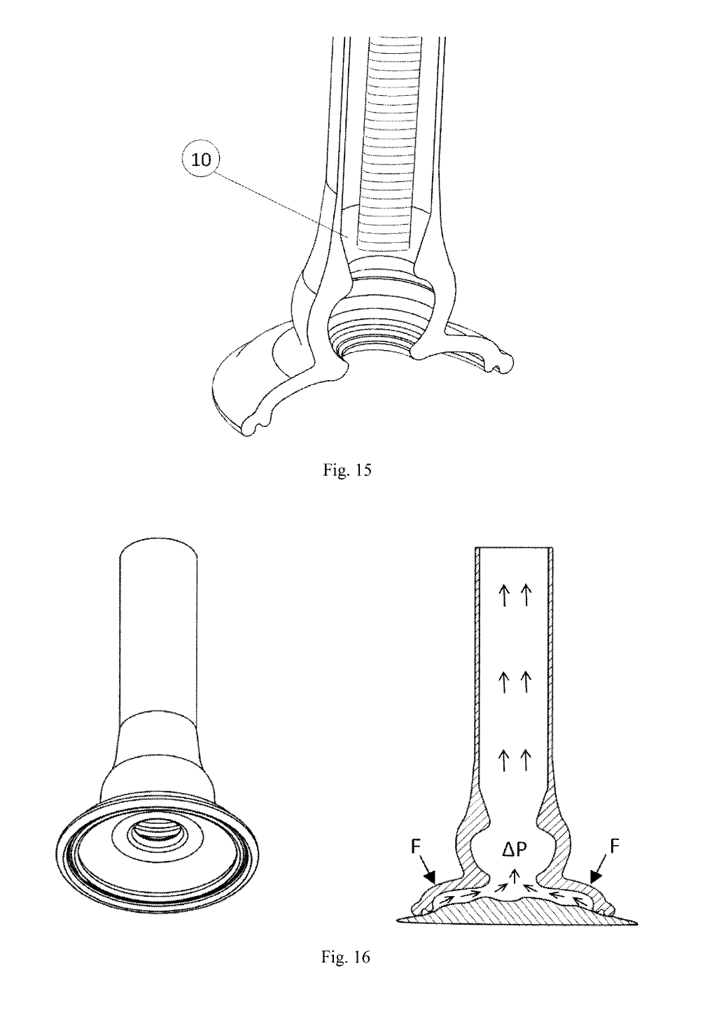

With reference now to FIG. 13, the vacuum chamber 12 of tip 100 maintains vacuum during use, and provides a reservoir of vacuum for the contact chamber 14. The wall thickness 4 of vacuum chamber 12 is preferably thicker than the wall 3 of contact chamber 14 to enhance its ability to withstand constant vacuum without collapsing. The wall 4 of vacuum chamber 12 may also be manufactured from a higher durometer material to further assist achieving this attribute.

The chambers divider lumen 13 connects the vacuum chamber 12 and contact chamber 14, and is suitably constructed and dimensioned to permit the free passage therethrough of a wire or dedicated occlusion penetrating device during use (see FIG. 14). In some embodiments, an additional lumen 18 may optionally be provided to run through the entire length of the catheter and extend all the way to the level of the distal tip for additional support of the wire or dedicated occlusion penetrating device 19.

The chambers divider recess 5 facilitates flexibility between the vacuum chamber 12 and contact chamber 14, thereby providing contact chamber 14 with additional degrees of freedom to bend and thus to better fit to the topography of the target surface without breaking vacuum, and also to minimize the effect of bending of the catheter shaft 16.

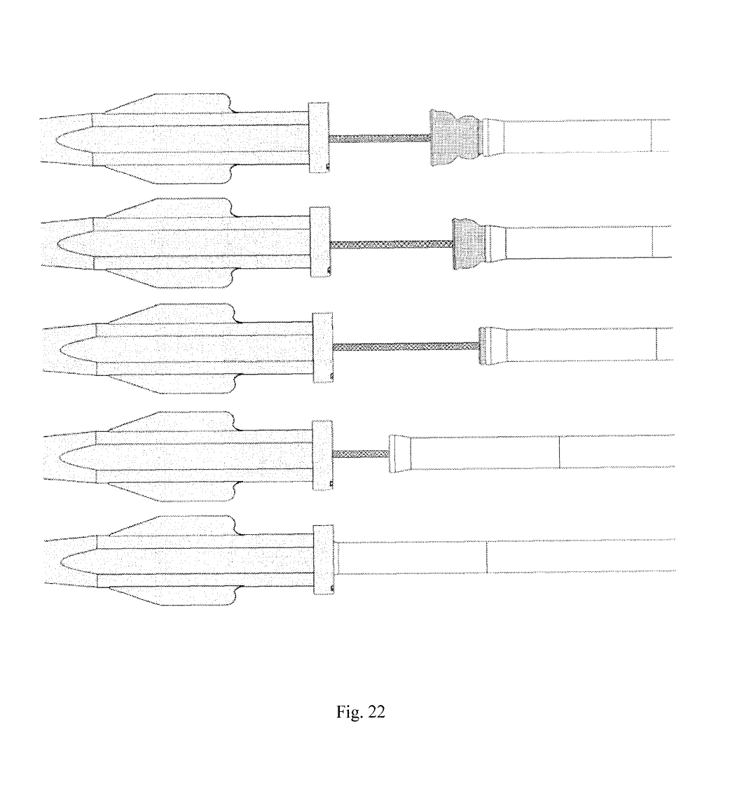

The vacuum chamber recess 6 provides a secondary flexibility zone, but also guides the tip 100 into its delivery sleeve prior to the procedure (see FIG. 21).

The tail 11 provides an interface between the flexible tip 100 and the catheter shaft 16, and it's the thickness and shape of the tail wall 7 are optimized for various known bonding or fusing techniques, including lamination, in which case tail wall 7 could be placed in between the layers that comprise a conventional catheter shaft 16.

Guiding cone 10 is dimensioned to guide the wire or dedicated occlusion penetrating device through the center of the tip 100, and reduces the risk of damage to the inner structure of tip 100 in embodiments where a stiff wire or dedicated occlusion penetrating device is being used (see FIG. 15).

Referring now to FIG. 16, the twin-chamber construction of tip 100 enables the more efficient maintenance of a stable level of vacuum as compared to prior known devices. Contact chamber 14 creates a robust sealing area, while the vacuum chamber 12 buffers and delivers a constant under-pressure “delta P” to maintain the adhering force “F”.

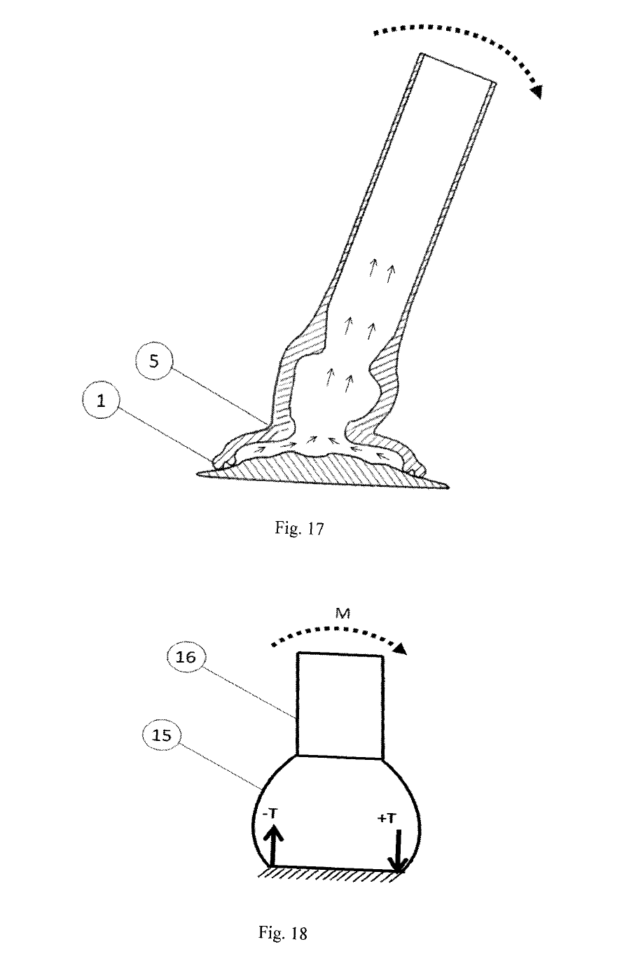

In addition, as best seen in FIG. 17, the twin-chamber construction of tip 100 and the hinge-like action of divider recess 5 enhances the ability of tip 100 to maintain contact chamber 14 generally parallel to the target surface despite changes in the inclination of catheter shaft 16. This further enhances the ability of the tip 100 to maintain a stable level of vacuum despite changes in the inclination of shaft 16, and isolates the contact chamber 14 from perturbations to the proximal portions of the catheter shaft 16.

By way of comparison, FIG. 18 illustrates the deleterious effects of bending on vacuum maintenance in a single chamber design. In such a single chamber design, if a bending force “M” is applied to the catheter shaft after vacuum has been built in single vacuum chamber 15, then the contact area of chamber 15 will experience compression (+T) and tension (−T) forces. Since the compression force assist in adhering to the contact surface, it is the tension force that needs to be minimized to prevent the contact area seal to break.

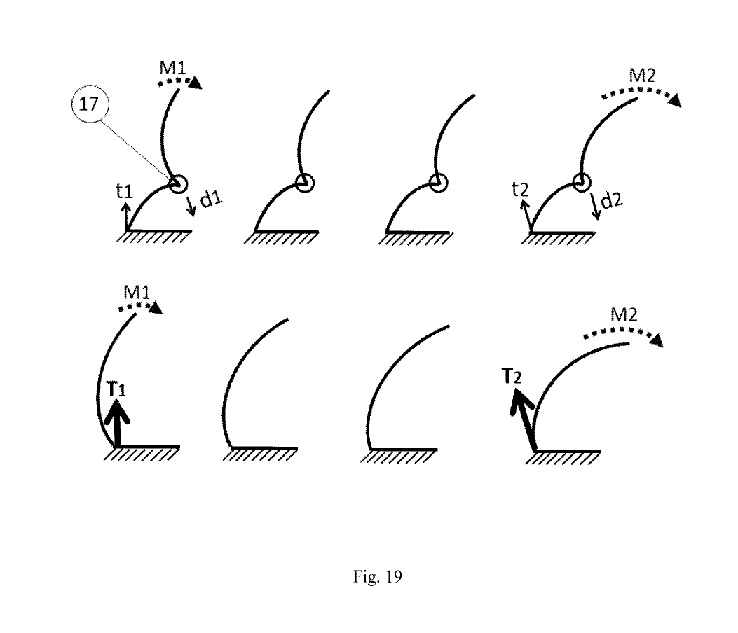

FIG. 19 illustrates these effects in greater detail vis-à-vis both a single vacuum chamber design 15 and the dual chamber design of the presently disclosed subject matter. In the dual chamber design, stress isolating point 17 (which, as described above, may comprise the twin-chamber construction of tip 100 and the hinge-like action of divider recess 5 of the present subject matter) results in a lower tension force (t1,t2) to be transmitted to the contact surface (sealing ring 1 and optionally also secondary sealing ring 8 of the present subject matter) as a consequence of shaft bending increments (M1, M2). In a single chamber design, such force increment (M1, M2) has higher effect on the tension magnitude (T1, T2) as compared to a dual chamber design. @M1: t1<T1; @M2: t2<<T2

The difference in force reaction is converted through the isolating point to different angled force vector (d1, d2), that causes internal deformation of the chambers which do not affect the tension force (t1, t2). @M1: t1+d1=T1; @M2: t2+d2=T2

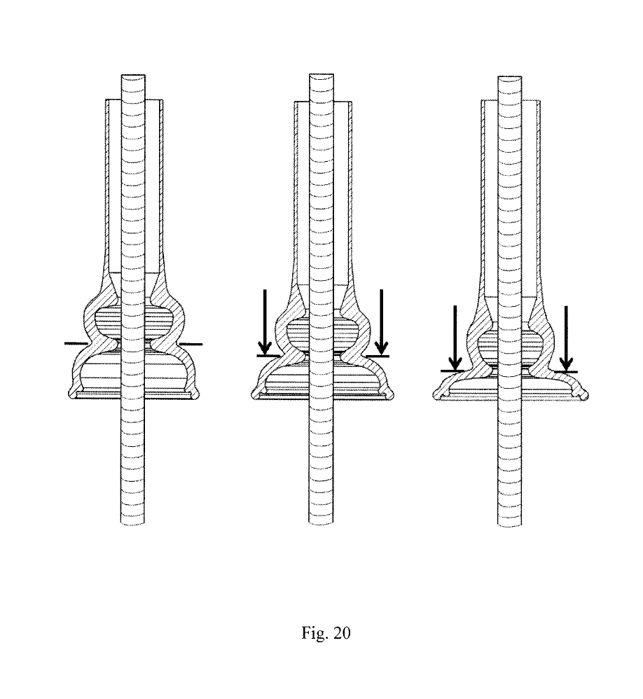

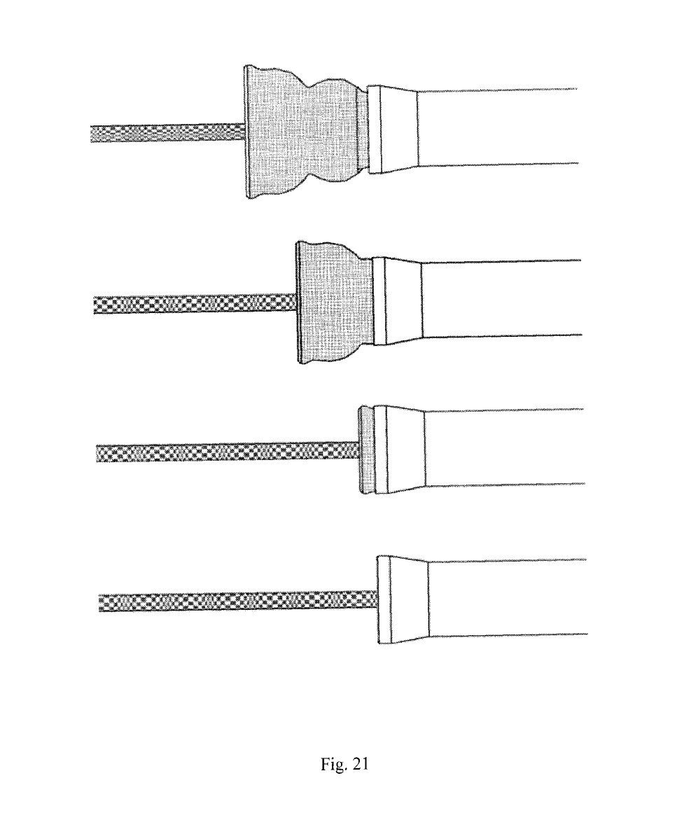

Referring now to FIG. 20, tip 100 permits the maintenance of a stable vacuum while allowing a wire or dedicated occlusion penetrating device to pass freely through lumen. Additionally, tip 100 it will not impose high drag to the wire or device during its passage regardless of the amount of vacuum. This is achieved by cooperation of the chambers divider 9 with vacuum chamber 13, such that radial deformation is minimized and compensated for by axial deformation upon vacuum actuation. This cooperative action keeps the chambers divider lumen 13 at an almost constant diameter regardless of the surrounding under-pressure, thereby permitting the free passage of the wire or device through to the target area.

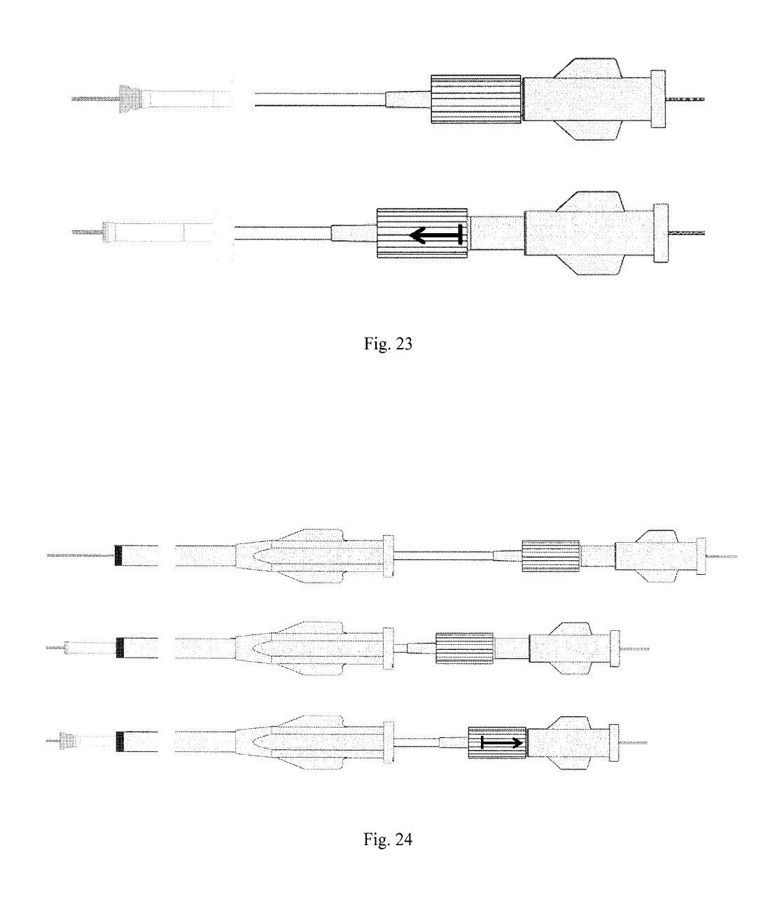

FIGS. 21 through 24 illustrate steps in the method of use of the vacuum anchoring catheter. Since the vacuum anchoring catheter is a percutaneous device, it is normally introduced via a guiding catheter, so its flared tip 100 tip should be compressed to enable loading into the guiding catheter lumen. One design for loading is a sliding sleeve connected to an actuating knob at the hub. The sleeve is pushed forward to capture the flared tip and encapsulate it to fit a smaller diameter to allow the vacuum anchoring catheter to be introduced into the guiding catheter (see FIG. 23). Once the vacuum anchoring catheter has reached the target occlusion, the sleeve using the knob is pulled back to expose the tip 100 to be ready for the occlusion penetrating procedure. Once the tip 100 makes contact with the target area of the occlusion, a vacuum is applied through the catheter by the withdrawal and temporary locking of a piston at the proximal end of the catheter. When the occlusion penetrating procedure is concluded, the vacuum is released and the guiding catheter is withdrawn (see FIG. 24).

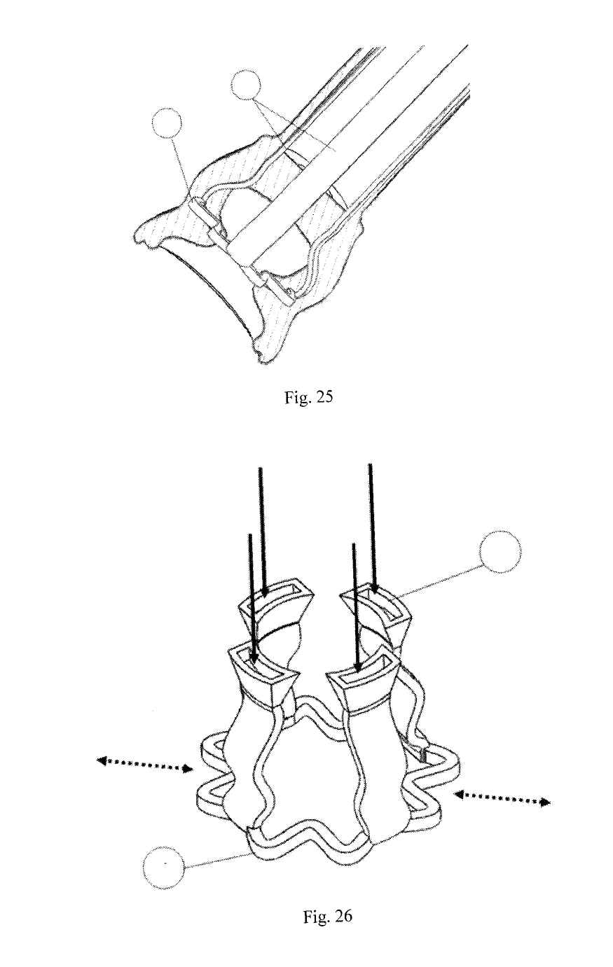

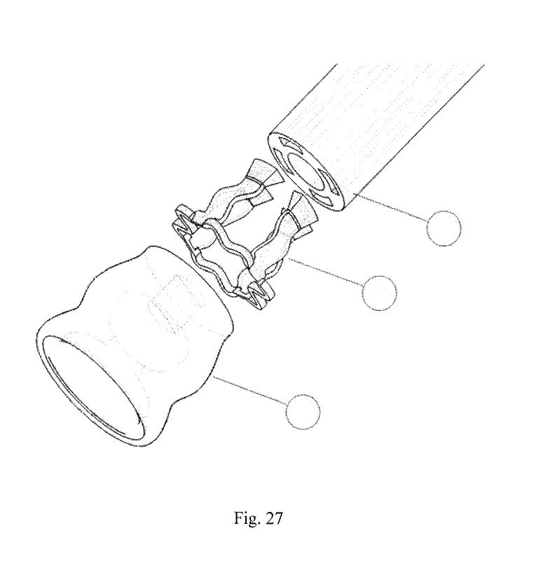

FIGS. 25 through 27 illustrate alternate embodiments in which vacuum is self-generated and continuously built by the bending movement of the catheter. In these embodiments, tip 100 further includes embedded spring frame 20 generally encircling chambers dividing lumen 13 and extending into catheter shaft 16. Bending of shaft 16 causes the spring frame 20 to convert the bending movement of the shaft 16 into radial expansion/contraction of the vacuum chamber wall 4, and thereby build vacuum by increasing/decreasing the volume of vacuum chamber 12.

In preferred embodiments, the frame 20 comprises radial spring 21 and two or more pairs of asymmetrical connecting struts 22 in communication with embedded actuation wires or struts 23 within the shaft 16. The embedded actuation wires or struts 23 within the shaft 16 are preferably located in dedicated lumens 24. In other embodiments, the frame may comprise an uneven number of connecting struts 22 and actuation wires or struts 23.

The present description includes the best presently contemplated mode of carrying out the subject matter disclosed and claimed herein. The description is made for the purpose of illustrating the general principles of the subject matter and not be taken in a limiting sense; the subject matter can find utility in a variety of implementations without departing from the scope of the disclosure made, as will be apparent to those of skill in the art from an understanding of the principles that underlie the subject matter.

We claim:

1. A catheter that uses vacuum enabled tip that improves the ability to deliver axial force through its lumen, due to its adherence to the contact surface.

2. A catheter for CTO devices that uses vacuum enabled tip that improves ability to deliver axial force through a CTO device to a blood vessel occlusion.

3. A catheter that by using its ability to adhere to the target surface with a vacuum, reduces the phenomena of catheter-sudden-pull-back that occurs when advancing a device through a catheter lumen which encounter an obstacle.

4. A catheter that can deliver a wire while maintain a vacuum throughout its lumen up and including its tip.

5. A catheter that can deliver a wire while maintaining a vacuum using the same lumen.

6. A catheter that can deliver a wire and maintain vacuum using two or more lumens.

7. A catheter tip that apply vacuum to a surface by using two joint chambers, were one chamber act as a “contact chamber” and deform to fit to the surface shape and the other a “vacuum chamber” maintain a relatively fixed sphere like shape to sustain the vacuum.

8. A catheter tip that provide vacuum by using two joint chambers and a “chambers divider septum”, to assist the “contact chamber” to deform and maintain vacuum as close as possible to the surface.

9. A catheter tip that uses two joint chambers and a “chambers divider recess” to minimize the pull stresses at the contact area due to bending of the shaft or bending of the tip proximal area.

10. A catheter tip that uses two joint chambers, a “chambers divider septum” and a “chambers divider recess” to partially or fully convert a shaft bending force into a pushing vertical force act on the “chambers divider septum” and the “contact chamber”.

11. A catheter tip that uses two joint chambers different from one another by shape.

12. A catheter tip that uses two joint chambers different from one another by wall thickness.

13. A catheter tip that uses two joint chambers different from one another by material properties.

14. A catheter tip that uses two joint chambers and one or more embedded sealing rings geometry shapes.

15. A catheter tip that uses two joint chambers to maintain vacuum for distal surface adhering, and can deliver a wire through its lumen without breaking the vacuum.

16. A catheter tip that uses two joint chambers, a “chambers divider septum” and a “chambers divider recess” to regulate, as a function of vacuum, the “chambers divider Lumen” diameter and keep it above a minimum diameter.

17. A catheter tip that uses two joint chambers to maintain vacuum for distal surface adhering, and can deliver a wire through its lumen without imposing high drag force on the wire regardless of the amount of vacuum being applied.

18. A catheter tip that uses two joint chambers to maintain vacuum for distal surface adhering, and provide a concentric guidance for a wire, by using a conical cavity.

19. A catheter with a flared tip that uses a sliding sleeve to encapsulate the tip with a protective layer and to reduce its diameter.

20. A catheter with a flared tip that uses a bi-directional sliding sleeve mechanism to encapsulate the tip and later release it back to its original shape and size.

21. A method for treatment of blood vessel occlusion comprising securing a guiding catheter during a procedure by temporarily adhering the tip of the guiding catheter to the treated area using a vacuum.

22. A guiding catheter with anchoring based on a self generated vacuum having a tip that continuously builds vacuum powered by the bending movement of the catheter shaft, wherein the tip comprises
i. a contact area;
ii. a vacuum generating chamber;
iii. a chambers divider; and
iv. a vacuum maintaining chamber.
